(19) United States
(12) Patent Application Publication
(10) Pub. No.: US 2023/0026291 A1
(43) Pub. Date: Jan. 26, 2023

(54) DETERMINING RISK OF CANCER RECURRENCE

(71) Applicant: TECHNOLOGICAL UNIVERSITY DUBLIN, Dublin (IE)

(72) Inventors: Aidan Meade, 7 Dublin (IE); Thi Nguyet Que Nguyen, 7 Dublin (IE)

(73) Assignee: TECHNOLOGICAL UNIVERSITY DUBLIN, Dublin (IE)

(21) Appl. No.: 17/788,520

(22) PCT Filed: Dec. 23, 2020
(86) PCT No.: PCT/EP2020/087845

§ 371 Date: Jun. 23, 2022

(30) Foreign Application Priority Data
Dec. 23, 2019 (GB) 1919174.1

(51) Int. Cl. G16H 50/30; G16H 50/20; G16H 30/40

(52) U.S. Cl. CPC G16H 50/30; G16H 50/20; G16H 30/40

(57) ABSTRACT

A method of determining risk of breast cancer recurrence in a patient has the steps: obtaining (304) hyperspectral imaging training data and known recurrence outcomes for the hyperspectral imaging training data; training (306) one or more neural networks using the hyperspectral imaging training data and corresponding known recurrence outcomes; obtaining (308) hyperspectral imaging patient data; and applying (310) the one or more neural networks to the hyperspectral imaging patient data so as to determine risk of cancer recurrence in the patient.

### FIELD OF INVENTION

The present invention relates to a method and apparatus for determining risk of cancer recurrence in a patient. In particular, the present invention has application in identifying breast cancer patients with low and intermediate risk of recurrence.

### BACKGROUND OF THE INVENTION

Breast cancers comprise 32% of all cancers and their incidence has increased by approximately 15% over the past 15 years, with 5-year survivorship at approximately 78%.

Breast cancer is a heterogeneous disease with a variable risk of recurrence. For the majority of patients who are diagnosed with endocrine receptor-positive (ER+) lymph node-negative (LN−) early stage breast cancer (ESBC), unnecessary treatment with chemotherapeutics is often prescribed after surgical resection, which is of no added clinical benefit but results in increased toxicity, leading to wastage of healthcare resources and higher post-treatment management costs. Clinical trials have failed to identify a set of biomarkers that segregate low or intermediate risk groups in these patients. The use of biomarkers for stratification of the risk of breast cancer tumour recurrence has only seen marked success in patients for whom therapeutic resistance or high recurrence rates are expected.

Predicting the likelihood of disease progression presents challenges for clinicians, particularly in patients where the disease is detected in the early stages.

The existing approaches for the stratification of ER+ LN− ESBC patients, on the basis of likelihood of future recurrence of the disease, include the Oncotype DX™ genomic assay (Genomic Health Inc.) which measures the gene expression levels of 16 cancer-related genes plus five control genes, the MammaPrint™ test (Agendia Inc.) which uses a 70 gene profile and OncoMasTR™ test which uses a combination of the expression levels of six master transcriptional regulators (i.e. FOXM1, UHRF1, PTTG1, E2F1, MYBL2 and HMGB2) and p16INK4a. Among the approaches, OncoType DX and MammaPrint have received FDA approval for clinical use and OncoMasTR is CE-marked and ISO accredited. These approaches are reported to have a very similar predictive power, with OncoTypeDX, MammaPrint and OncoMasTR having area-under-curve values of 0.69, 0.59 and 0.69 respectively, as reported in Lanigan et al., “Delineating transcriptional networks of prognostic gene signatures refines treatment recommendations for lymph node-negative breast cancer patients” Febs j 282, pp. 3455-3473 (2005) and Goldstein et al., “Prognostic utility of the 21-gene assay in hormone receptor-positive operable breast cancer compared with classical clinicopathologic features” J Clin Oncol 26, pp. 4063-4071 (2008).

The reported results indicate that the aforementioned approaches fail to identify a significant proportion of patients at lower risk of cancer recurrence, for whom chemotherapy is unnecessary.

Furthermore the aforementioned approaches are expensive and have long turnaround times (approximately two weeks), causing additional burden on the patients. Accurately predicting risk of recurrence in these patients is therefore of significant importance.

Chemical imaging, via either Raman or Fourier Transform Infrared (FTIR) microspectroscopy, is a non-invasive diagnostic tool for cancer histopathology. The FTIR spectrum of a molecule comprises the frequencies of the modes of vibration of all of the organic bonds within the sample that may be excited through transmission of infrared light through the sample. The FTIR spectrum is altered as a result of pre-translational (DNA methylation) and post-translational effects (phosphorylation, acetylation, glycosylation, etc.) in disease states. Chemical imaging uses this spectral information to objectively identify tissue biochemistry without the use of extraneous tissue labeling, such that objective histopathological classification models may be constructed.

The advent of deep-learning techniques offers a pipeline directly from image acquisition to classification either via digital pathology or whole image classification. Prior art has demonstrated the capability of deep-learning techniques in detecting abnormalities in cells, including cancer in skin tissue cells with RGB (red, green and blue) image data as disclosed in Esteva et al., “Dermatologist-level classification of skin cancer with deep neural networks”, Nature 542, pp. 115-118, (2017).

Deep-learning neural networks are known for classification of individual spectral data in discrimination of individual chemical species as disclosed in Liu et al., “Deep convolutional neural networks for Raman spectrum recognition: a unified solution”, Analyst 142, pp. 4067-4074 (2017).

Deep-learning neural networks have been used to classify FTIR images of tissue samples (for example, as disclosed in Berisha et al., “Deep learning for FTIR histology: leveraging spatial and spectral features with convolutional neural networks”, Analyst 144, pp. 1642-1653, (2019)). However, in these cases, extensive pre-processing steps were employed before input of data to the networks. This limits the power of spectral data from providing rich biochemical and spectro-morphological information.

In summary, prior art techniques for determination of ER+ LN− ESBC lack sensitivity and accuracy, leading to additional unnecessary testing and expensive procedures.

### SUMMARY OF INVENTION

There is a need for a method and associated apparatus which produces an unambiguous result and minimises turnaround time with reduced costs. It is desirable to provide an improved method and apparatus for determining risk of breast cancer recurrence, which overcomes at least some of the above-identified problems and allows low- and intermediate-risk early-stage breast cancer patients to be stratified in an accurate, cost-effective and non-invasive manner.

According to a first aspect of the present invention, there is provided a method of determining risk of cancer (typically cancer recurrence) in a patient, the method comprising the steps:
obtaining hyperspectral imaging training data and known recurrence outcomes for the hyperspectral imaging training data;
training one or more neural networks using the hyperspectral imaging training data and corresponding known recurrence outcomes;
obtaining hyperspectral imaging patient data; and
applying the one or more neural networks to the hyperspectral imaging patient data so as to determine risk of cancer (typically cancer recurrence) in the patient.

Preferably, the cancer is an epithelial cancer. In one embodiment, the cancer is breast cancer. In one embodiment, the cancer is a recurring cancer (local, regional or distant recurrence)—examples apart from breast cancer include prostate, ovarian, lymphoma, glioblastoma, bladder, colorectal, thyroid, pancreas, melanoma, and leukemia. Other cancers include multiple myeloma, prostate cancer, glioblastoma, lymphoma, fibrosarcoma; myxosarcoma; liposarcoma; chondrosarcoma; osteogenic sarcoma; chordoma; angiosarcoma; endotheliosarcoma; lymphangiosarcoma; lymphangioendotheliosarcoma; synovioma; mesothelioma; Ewing's tumour; leiomyosarcoma; rhabdomyosarcoma; colon carcinoma; pancreatic cancer; breast cancer; node-negative, ER-positive breast cancer; early stage, node positive breast cancer; early stage, node positive, ER-positive breast cancer; ovarian cancer; squamous cell carcinoma; basal cell carcinoma; adenocarcinoma; sweat gland carcinoma; sebaceous gland carcinoma; papillary carcinoma; papillary adenocarcinomas; cystadenocarcinoma; medullary carcinoma; bronchogenic carcinoma; renal cell carcinoma; hepatoma; bile duct carcinoma; choriocarcinoma; seminoma; embryonal carcinoma; Wilms' tumour; cervical cancer; uterine cancer; testicular tumour; lung carcinoma; small cell lung carcinoma; bladder carcinoma; epithelial carcinoma; glioma; astrocytoma; medulloblastoma; craniopharyngioma; ependymoma; pinealoma; hemangioblastoma; acoustic neuroma; oligodendroglioma; meningioma; melanoma; retinoblastoma; and leukemias.

Preferably, the hyperspectral imaging patient data is obtained from a sample obtained from the patient (i.e. in-vitro analysis), typically a tissue sample. In another embodiment, the hyperspectral imaging patient data is obtained in-vivo, for example using suitable imaging techniques.

Preferably the hyperspectral imaging patient data is obtained using an infra-red imaging device, preferably a FTIR IR imaging device.

In another embodiment, the hyperspectral imaging patient data is obtained using quantum cascade laser imaging.

In yet another embodiment the hyperspectral imaging patient data is obtained using Raman imaging, coherent anti-Stokes Raman imaging or stimulated Raman imaging.

Preferably, the one or more neural networks are deep-learning convolutional neural networks (DL-CNN). In one embodiment, the at least one neural network (typically the at least one DL-CNN) is configured to receive hyperspectral imaging data as an input.

Preferably, the steps of obtaining hyperspectral imaging training and patient data comprise measuring biopsy samples from patients to generate the respective hyperspectral imaging training and patient data

Preferably, the method of further can comprise the step of constructing full face tissue sections or tissue microarray blocks from the biopsy samples from the patients.

Preferably, the method can also comprise the use of hyperspectral imaging data from formalin-fixed paraffin preserved tissue specimens where said tissue may or may not be de-waxed chemically.

Preferably, the step of training one or more neural networks using the hyperspectral imaging training data comprises inputting the hyperspectral imaging training data as an input to the first layer of one or more of the neural networks.

Preferably, the hyperspectral imaging data comprise spatial information and spectral information.

Preferably, the hyperspectral imaging data comprise raw spectral data.

Preferably, the hyperspectral imaging data comprise spectral data corresponding to a plurality of hyperspectral variables.

Preferably, the plurality of hyperspectral variables comprise more than three hyperspectral variables.

Preferably, the plurality of hyperspectral variables comprise more than 700 hyperspectral variables.

Preferably, the step of training the one or more neural networks comprises the steps:
using a first portion of the hyperspectral imaging training data and the corresponding known recurrence outcomes to adjust weights of the neural networks so as to produce one or more trained neural network; and
inputting a second portion of the hyperspectral imaging training data to the one or more trained neural network to validate the performance of the trained neural network in reproducing the corresponding known recurrence outcomes so as to identify a validated neural network,

and wherein the step of applying the one or more neural networks to the hyperspectral imaging patient data comprises applying the validated neural network to the hyperspectral imaging patient data so as to determine risk of cancer recurrence in the patient.

Preferably, the step of applying the one or more neural networks to the hyperspectral imaging patient data comprises:

Inputing to a first convolution layer, images which have a predetermined dimension;

filtering the image using a plurality of first convolution layer kernels to extract features from the input image;

Sub-sampling the image to create a plurality of first feature maps;

inputing the plurality of first feature maps into a second convolution layer and filtering the feature maps using a plurality of second convolution layer kernels;

sub-sampling the output of the second convolution layer to create a plurality of second feature maps; wherein

the second feature maps is input to at least one first fully connected layer which produces a binary classification on the basis of the outputs of all of the preceding layers and which predicts recurrence or non-recurrence.

Preferably, the image is a chemical image.

Preferably, the image has a dimension of 256×256 pixels in the x-y direction and 106 wavenumbers in the z-direction.

Preferably, the step of filtering the image using a plurality of first convolution layer kernels uses a layer stride is 1×1 pixel along the x-y dimension

Preferably, the step of sub-sampling the image to create a plurality of first feature maps uses a max pooling layer of 2×2 pixels.

Preferably, the second feature maps is processed by a first and second fully connected layer.

Preferably, the first and second fully connected layers have 180 and 100 neurons, respectively.

Preferably, each fully connected layer is followed by a dropout layer.

Preferably the dropout layer has a frequency of rate 0.5 for preventing overfitting during training.

Preferably a rectified linear unit function is used as activation function for the output of each CL and FCL within the complete neural network.

Preferably, the step of training and validating one or more neural network comprises:

Receiving imaging data in which recurrence of a condition was seen;

splitting the data randomly by patient into training, validation, and test data sets training the network through a predetermined number of epochs to create a plurality of models and identifying an optimal model from the models as being with model with the highest value of area under a receiver operating characteristic curve of validation.

According to a second aspect of the present invention, there is provided a risk determination apparatus for determining risk of cancer recurrence in a patient, the risk determination apparatus comprising:
a data measurement system configured to measure hyperspectral imaging patient data;
one or more neural networks trained using hyperspectral imaging training data and corresponding known recurrence outcomes; and
a processor configured to:
   receive the hyperspectral imaging patient data;
   apply the one or more neural networks to the hyperspectral imaging patient data so as to determine risk of cancer recurrence in the patient.

According to a third aspect of the present invention, there is provided computer program product comprising a computer usable medium, where the computer usable medium comprises a computer program code that, when executed by a computer apparatus, determines risk of cancer recurrence in a patient according to the method of the first aspect.

The invention also provides a method of treating a patient identified as being at risk of cancer, or cancer recurrence, according to a method of the invention, the method comprising the steps applying the method of the invention to the patient, and when the method identifies a risk of cancer, administering a therapy to the patient, generally a prophylactic therapy.

Other aspects and preferred embodiments of the invention are defined and described in the other claims set out below.

### BRIEF DESCRIPTION OF DRAWINGS

Embodiments of the present invention will now be described, by way of example only, with reference to the drawings, in which:

FIG. 1 illustrates, in schematic form, a prior art Le-Net5 DL-CNN architecture taking a 2D image as an input.

FIG. 2 illustrates, in schematic form, a modified Le-Net5 DL-CNN architecture configured to receive hyperspectral imaging data as an input.

FIG. 3 is a flowchart illustrating a method of determining risk of cancer recurrence in a patient, in accordance with an embodiment of the present invention.

FIG. 4 is a flowchart illustrating a process of training six DL-CNN architectures using the method of FIG. 3.

FIG. 5 is a chart illustrating the performance of the six DL-CNN architectures.

FIG. 6 illustrates, in schematic form, a risk determination apparatus for determining risk of cancer recurrence in a patient, in accordance with an embodiment of the present invention.

FIG. 7 illustrates, in schematic form, a DL-CNN architecture configured to receive hyperspectral imaging data as an input.

FIG. 8(a) is a graph showing Network biases for the first convolutional layer of the neural network depicted in FIG. 7.

FIG. 8(b) is a graph showing Kernel weights for the first convolutional layer of the neural network depicted in FIG. 7.

FIG. 9(a) is a graph showing network biases for the second convolutional layer of the neural network depicted in FIG. 7.

FIG. 9(b). Kernel weights for the second convolutional layer of the neural network depicted in FIG. 7.

FIG. 10 illustrates in schematic form a method of the invention.

### DESCRIPTION OF EMBODIMENTS

All publications, patents, patent applications and other references mentioned herein are hereby incorporated by reference in their entireties for all purposes as if each individual publication, patent or patent application were specifically and individually indicated to be incorporated by reference and the content thereof recited in full.

Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, age, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, an effective amount or a therapeutically effective amount of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure. Improvement may be observed in biological/molecular markers, clinical or observational improvements. In a preferred embodiment, the methods of the invention are applicable to humans, large racing animals (horses, camels, dogs), and domestic companion animals (cats and dogs).

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, camels, bison, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human. As used herein, the term “equine” refers to mammals of the family Equidae, which includes horses, donkeys, asses, kiang and zebra.

“Hyperspectral imaging data” or “HIS data” is a hybrid modality that combines imaging and spectroscopy and comprises collecting spectral information at every pixel of as two-dimensional (2-D) detector array and generation of a three-dimensional (3-D) dataset of spatial and spectral information, known as a hypercube. In the 3-D hypercube, the first two dimensions (x-axis and y-axis) generally contain the spatial dependence of the data and the third dimension (z-axis) contains the spectral dependence of the data.

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

Deep-learning techniques involve an application of neural networks with a number of hidden layers to internally identify patterns in data without the need for feature engineering.

A multilayer neural network typically involves a plurality of artificial neurons arranged in layers including an input layer, an output layer and one or more hidden layers in between. An input pattern is described by a number of neurons within the input layer, where each neuron is associated with a weight. The weighted sum of the neurons generates an output signal, which is successively propagated between hidden layers to transform the input pattern into an output pattern computed by an output function (e.g. step function or sigmoid function). The objective of the neural network is to enable the computed output pattern to closely approximate the expected output pattern, so that when a test input is provided, the corresponding output pattern can be derived. This is achieved by training the neural network with a number of input patterns and corresponding expected output patterns, wherein a learning algorithm is used to adjust the weights associated with the neurons of the network so that a relationship between the input and output patterns can be captured. Such learning process is an iterative process, which can be time-consuming and resource-intensive.

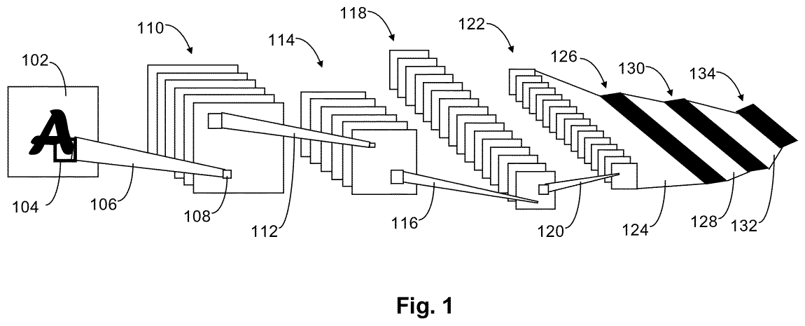

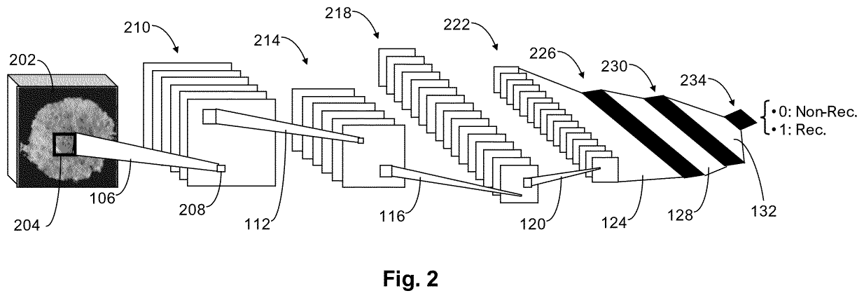

In the Figures, elements labeled with reference numerals found in the preceding Figures represent the same elements as described for the respective preceding Figure. For example, feature 106 in FIG. 2 corresponds to the same feature 106 as described with reference to FIG. 1.

A prior art LeNet-5 deep-learning convolutional neural network (DL-CNN), as described in LeCun et al., “Gradient-based learning applied to document recognition”, Proceedings of the IEEE 86, pp. 2278-2324 (1998), is an example of a multilayer neural network for predicting handwritten digits.

With respect to FIG. 1, the LeNet-5 DL-CNN architecture is implemented using a combination of a) convolutional operations 106 and 116 for extracting features (e.g. 108) from an input 102 and various layers of the architecture; b) sub-sampling operations 112 and 120 for reducing dimensions of feature maps; and c) full connection operations 124, 128 and 132 for classification of the input.

Referring to FIG. 1 in more detail, the prior art LeNet-5 DL-CNN architecture comprises seven layers as follows.

Layer 110, which is connected to an input of a 32×32 pixel image 102, is a convolutional layer with 6 feature maps, wherein each of the feature maps is a 28×28 pixel image;

Layer 114 is a sub-sampling layer with 6 feature maps, each of which is a 14×14 pixel image;

Layer 118 is a convolutional layer with 16 feature maps, each of which is a 10×10 pixel image;

Layer 122 is a sub-sampling layer with 16 feature maps, each of which is a 5×5 pixel image;

Layer 126 is a fully-connected convolutional layer with 120 units, each of which is connected to all the 400 (5×5×16) nodes in the layer 122;

Layer 130 is a fully-connected layer with 84 units, each of which is fully connected to all the 120 nodes in the layer 126; and

Layer 134 is an output layer containing 10 classification results.

Unlike RGB imaging, which captures three spectral bands (Red, Green and Blue) in the light spectrum, hyperspectral imaging may collect hyperspectral data characterised by a wide range of hyperspectral variables and at the same time records spatial information in an image. Hyperspectral imaging data are generally presented in a 3D cube where the first two dimensions (x-axis and y-axis) contain the spatial dependence of the data and the third dimension (z-axis) contains the spectral dependence of the data.

The hyperspectral imaging data requires more complex data processing than RGB imaging data. Previously, few neural networks have been operated to receive hyperspectral imaging data as an input, nor have any visualization methods been applied to discover spectral and morphological (or chemical-pathological) features learnt by these neural networks.

Embodiments of the present invention allow a DL-CNN architecture to be coupled with hyperspectral imaging data, wherein extensive pre-processing of the hyperspectral imaging data before being fed into the DL-CNN architecture is not required.

Embodiments of the present invention operate on samples which are not chemically de-waxed after normal pathological preservation, leaving the sample available for other analyses hyperspectral imaging is non-destructive and label free.

FIG. 2 illustrates a modified LeNet-5 DL-CNN architecture specifically designed for receiving the hyperspectral imaging data as an input. In this example, the hyperspectral imaging data represent a number of 106 2D images 202, each of which contains spectral data 204 corresponding to individual hyperspectral variables. The modified LeNet-5 DL-CNN architecture is configured with the following layers:

Layer 210, which is connected to an input of a 256×256×106 image 202, is a convolutional layer with 12 feature maps, wherein each of the feature maps has a dimension of 5×5×106 and contains extracted features (e.g. 208); the stride in this layer is 1×1 pixel along the x-y direction;

Layer 214 is a sub-sampling layer with 2×2 feature maps, producing thus 12 feature maps with a dimension of 126×126 pixels;

Layer 218 is a convolutional layer with 25 kernels each of which has dimension 5×5×12;

Layer 222 is a sub-sampling layer with 2×2 feature maps and a stride of 1×1 along the x-y dimension, producing thus 25 feature maps of 61×61 pixels;

Layer 226 is a fully-connected convolutional layer with 180 units, each of which is connected to the layer 122;

Layer 230 is a fully-connected layer with 100 units, each of which is fully connected to all the 180 nodes in the layer 226; and

Layer 234 is an output layer containing two classification results including 0 and 1, which indicate, for example, non-recurrence (0) or recurrence (1) in ER+ LN− ESBC patients with low and intermediate risk of recurrence.

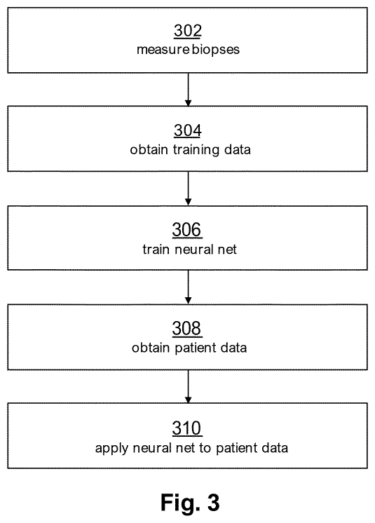

FIG. 3 illustrates a flowchart of a method of determining risk of cancer recurrence in a patient, in accordance with an embodiment of the present invention. The method has the following steps.

At step 302, a biopsy sample from a patient is measured to obtain hyperspectral imaging patient data. This step may comprise formalin fixation and paraffin preservation of the tissue from the patient and sectioning of said tissue onto a slide for imaging purposes.

At step 304, hyperspectral imaging training data are obtained from patients with known recurrence outcomes. This step may comprise constructing tissue microarray blocks from biopsy samples of the patients to generate the hyperspectral imaging training data. The step may involve the cutting of a 5 μm section of the tissue and mounting on a calcium fluoride substrate. The acquisition of hyperspectral or chemical images may involve inserting the sample into the focus of a Fourier-transform infrared microscope and acquiring images of the sample with 5.5 μm² over the spectral range from 1000-1800 cm⁻¹ at a 16 cm⁻¹ spectral resolution, with 4 scans per pixel and 2×2 tiles per image.

At step 306, one or more neural networks using the hyperspectral imaging training data and corresponding known recurrence outcomes are trained. This step may comprise a) using a first portion of the hyperspectral imaging training data and the corresponding known recurrence outcomes to adjust weights of the neural networks so as to produce one or more trained neural networks; and b) inputting a second portion of the hyperspectral imaging training data to the one or more trained neural network to validate the performance of the one or more trained neural networks in reproducing the corresponding known recurrence outcomes so as to identify a validated neural network.

At step 308, hyperspectral imaging patient data are obtained from a patient. This step may comprise formalin fixation and paraffin preservation of the tissue from the patient and sectioning of said tissue onto a slide for imaging purposes.

At step 310, the one or more (optionally validated) neural networks are applied to the hyperspectral imaging patient data so as to determine risk of cancer recurrence in the patient.

With reference to FIG. 3, the step 310 of applying the one or more neural networks to the hyperspectral imaging patient data may comprise applying the validated neural network to the hyperspectral imaging patient data so as to determine risk of cancer recurrence in the patient.

In this example, the hyperspectral imaging training data and the hyperspectral imaging patient data comprise spatial information and spectral information. The hyperspectral imaging training data and the hyperspectral imaging patient data in this example comprise raw spectral data, corresponding to a plurality of hyperspectral variables (e.g. three channels). The plurality of hyperspectral variables may comprise more than three hyperspectral variables, preferably up to or equal to 700 hyperspectral variables, or over 700 hyperspectral variables.

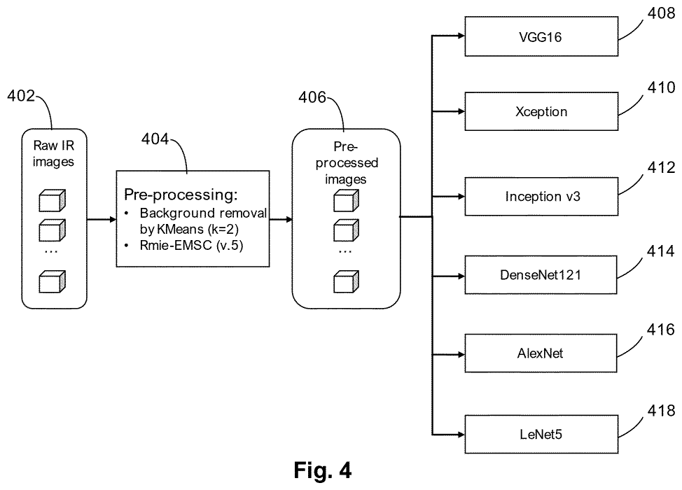

FIG. 4 illustrates an example process using an embodiment of the present invention (steps 304 and 306 of FIG. 3) to train six DL-CNN architectures. The six DL-CNN architectures are based on respective prior art DL-CNN architectures, including VGG16, Xception, Inception v3, DenseNet121, AlexNet and LeNet-5, and are specifically adapted to receive the hyperspectral imaging data as an input. The example process has the following steps.

At step 404, non-extensive pre-processing techniques are applied to at least some of raw hyperspectral imaging data 402 to generate pre-processed hyperspectral imaging data 406. This step may comprise using the K-Means clustering algorithm to remove background noise and the RMieS-EMSC algorithm to correct spectral distortion in the raw hyperspectral imaging data 402. The RMieS-EMSC is described in P. Bassan et al., “Resonant Mie Scattering (RMieS) correction of infrared spectra from highly scattering biological samples”, Analyst, 135, pp. 268-277 (2010). Non-extensive pre-processing of the raw hyperspectral imaging data 402 enables rich biochemical and spectro-morphological information captured in the raw hyperspectral imaging data 402 to be retained and analysed, which contribute to accurate differentiation of abnormal (e.g. cancerous) tissues from normal tissues.

At steps 408-418, the six DL-CNN architectures are trained at the respective step using the pre-processed hyperspectral imaging data 406 and known recurrence outcomes. In another example, the six DL-CNN architectures are trained using at least some of the raw hyperspectral imaging data.

With respect to the steps 408-418, training the DL-CNN architectures may comprise a) using a first portion of the pre-processed hyperspectral imaging data 406 and corresponding known recurrence outcomes to adjust weights of the respective DL-CNN architectures so as to produce respectively trained DL-CNN architectures; and b) inputting a second portion of the pre-processed hyperspectral imaging data 406 to the respectively trained DL-CNN architectures to validate the performance of the respectively trained DL-CNN architectures in producing corresponding known recurrence outcomes so as to identify respectively validated DL-CNN architectures.

With respect to FIG. 4, FTIR spectroscopic imaging of tissue microarray (TMA) blocks can be used to obtain the raw hyperspectral imaging data 402. In an example, the TMA blocks are constructed on biopsy samples acquired from 144 patients at resection prior to chemotherapy. The biopsy samples are obtained from a breast cancer patient cohort as described in DeNardo et al., “Leukocyte complexity predicts breast cancer survival and functionally regulates response to chemotherapy” Cancer Discov 1, pp. 54-67 (2011) and Mulrane et al., “miR-187 is an independent prognostic factor in breast cancer and confers increased invasive potential in vitro”, Clin Cancer Res 18, pp. 6702-6713 (2012). Among the 144 patients, 29 patients exhibited recurrence of breast cancer within 5 years of treatment. A 5 μm thick section of the TMA blocks was cut and mounted on calcium-fluoride slides for chemical imaging without chemical dewaxing. In this example, an Agilent Cary 630 FTIR micro-spectrometer is deployed to generate the raw hyperspectral imaging data 402 from the TMA blocks, which uses a spectral range from 1000-1800 cm⁻¹ with 16 cm⁻¹ resolution, 4 scans per pixel and 2×2 tiles per image. Scan time required for each image was approximately 1 min.

Referring to FIG. 4 in more detail, the pre-processed hyperspectral imaging data 406 is split randomly into the first portion and the second portion in a stratified manner with an 80%:20% split, and performance of the six DL-CNN architectures were evaluated over 500 epochs.

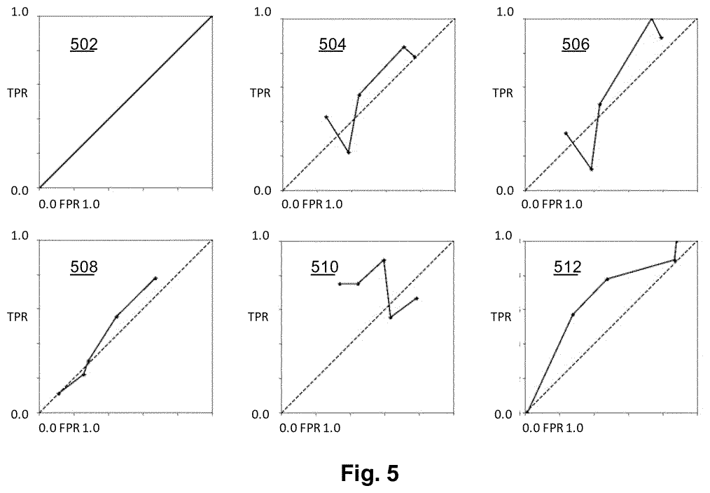

FIG. 5 indicates typical ROC (receiver operating characteristic) curves (502-512) for the six DL-CNN architectures corresponding to VGG16, Xception, Inception v3, DenseNet121, AlexNet and LeNet-5 DL-CNN architectures, respectively, wherein AUC values (an area under the curve) of the respective ROC curves (502-512) are calculated to be 0.51±0.03, 0.52±0.03, 0.58±0.05, 0.54±0.05, 0.48±0.02 and 0.64±0.07. The ROC curve plots the true positive rate (TPR) against the false positive rate (FPR).

With respect to FIG. 4, the six DL-CNN architectures are trained using the hyperspectral variables of the pre-processed hyperspectral imaging data 406.

FIG. 4 illustrates that, among all the trained DL-CNN architectures, the modified LeNet-5 DL-CNN architecture, as illustrated in FIG. 2, generated the greatest AUC of 0.64 which matches well the performance of the prior art approaches including OncoType-DX (AUC: 0.69), MammaPrint (AUC: 0.59) and OncoMasTR (AUC: 0.69) as stated earlier. The modified LeNet-5 DL-CNN architecture is demonstrated to be sufficient for determining risk of recurrence of ER+ LN− ESBC patients, wherein extraneous labels and signal enhancement are not required.

Embodiments of the present invention provide a specially purposed DL network incorporating a large number of hyperspectral variable channel inputs deliver a label-free chemical imaging-AI platform that significantly enhances breast cancer management.

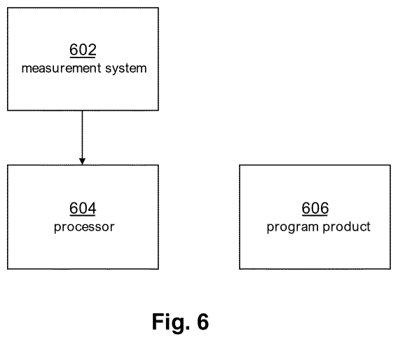

FIG. 6 illustrates a risk determination apparatus for determining risk of cancer recurrence in a patient comprising a) a data measurement system 602 configured to measure hyperspectral imaging patient data; b) one or more neural networks trained using hyperspectral imaging training data and corresponding known recurrence outcomes; c) a processor 604 configured to receive the hyperspectral imaging patient data and apply the one or more neural networks to the hyperspectral imaging patient data so as to determine risk of cancer recurrence in the patient.

In an example, the one or more neural networks comprise the modified LeNet-5 DL-CNN architecture of FIG. 2.

A computer program product 606 is illustrated in FIG. 6. It comprises a computer usable medium, where the computer usable medium comprises a computer program code that, when executed by a computer apparatus (such as processor 604), determines risk of cancer recurrence in a patient according to the method described with reference to FIGS. 2 to 4. In an example, the computer program code for each of the DL-CNN architectures was imported into Python (v. 3.7) incorporating Keras (v. 2.2.4) and TensorFlow (v.1.14.0) libraries.

Description of DL-Convoluted Neural Network

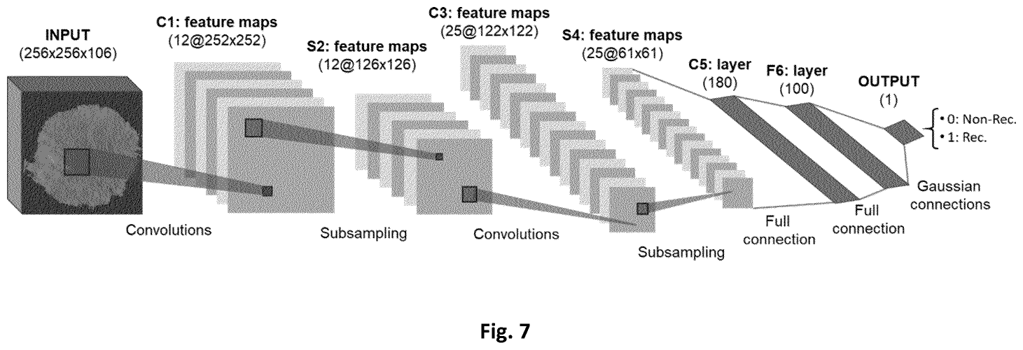

The network architecture allows the input of chemical images with a dimension of 256×256 pixels in the x-y direction and 106 wavenumbers in the z-direction and is depicted in FIG. 7.

The network consists of 2 convolutional layers (CLs) and 2 fully connected layers (FCLs). The first CL filters the input spectral image with dimension 256×256×106 using 12 kernels, where each kernel has a dimension of 5×5×106 pixels. In this layer the stride is 1×1 pixel along the x-y dimension. This CL is followed by a max pooling layer of 2×2 pixels for subsampling, producing thus 12 feature maps with a dimension of 126×126 pixels. These feature maps are input to the second CL which uses 25 kernels of size 5×5×12. The output of this layer is then input to another max pooling layer in which the filter has dimension of 2×2 pixels with a stride of 1×1, thus producing 25 feature maps of 61×61 pixels. The output of this layer is then directed through two FCLs. The first and second FCLs have 180 and 100 neurons, respectively. Each FCL is followed by a dropout layer with a frequency of rate 0.5 for preventing overfitting during training. The rectified linear unit function is used as activation function for the output of each CL and FCL within the complete neural network.

The output layer then produces a binary classification on the basis of the outputs of all of the preceding layers. As the network produces a binary classification, the output layer is a single neuron layer with sigmoid activation function that produces a value between 0 and 1. If this value is 1 then the prediction is Recurrence; otherwise, it is Non-Recurrence. Here binary cross-entropy is used as a loss function coupled with Adam optimization.

To train and validate this network, FTIR chemical imaging data were obtained from a cohort of 142 Swedish breast cancer patients in which 29 saw recurrence of the disease [1,2]. This data set was split randomly by patient into training, validation, and test sets in a manner that the training and validation sets had a balanced number of patients for both classes, with a proportion of 60%:20%:20% split on the class that has the smallest number of patients (i.e., Recurrence class). The whole network was trained and validated through 500 epochs. The model with the highest value of area under the receiver operating characteristic curve of validation is saved as the optimal model.

The performance of the network was test and evaluated over 5 independent runs.

The biases and weights of the optimized network were saved as .hdf5 files and visualised with HDFViewer v.3.1.2.

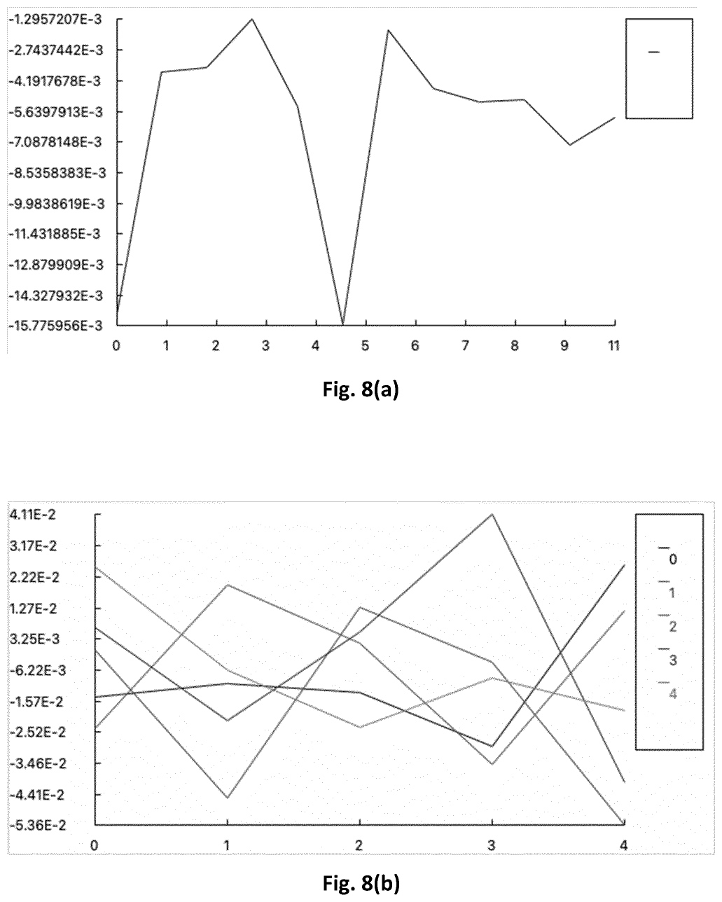

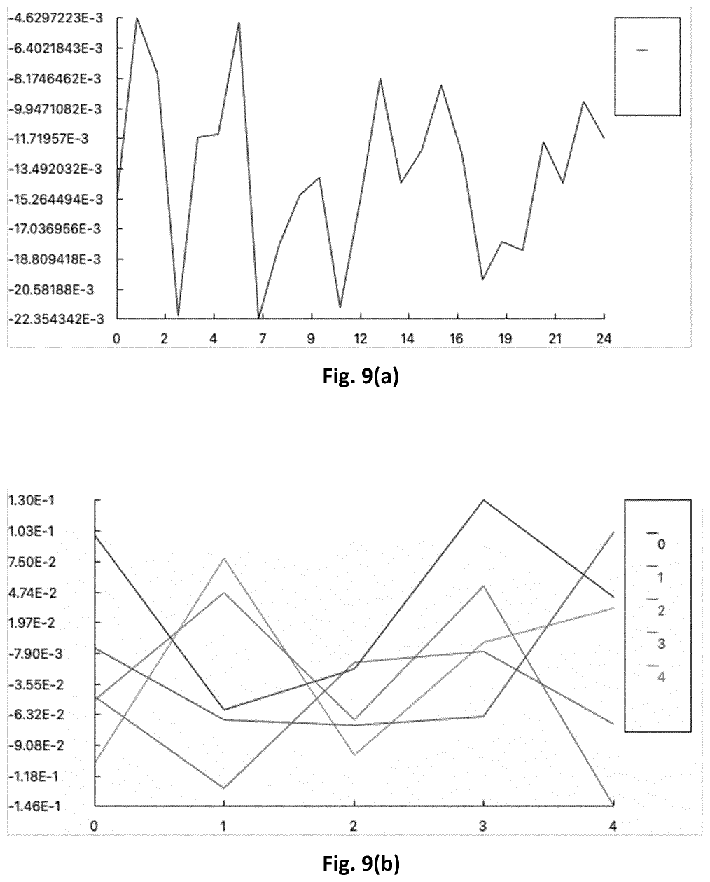

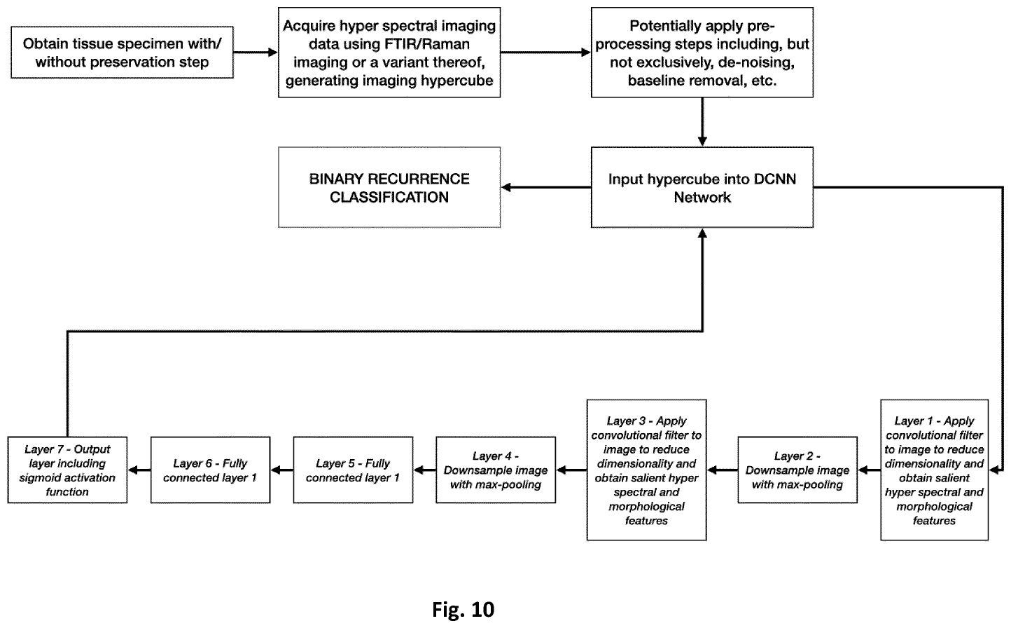

For the first convolutional layer mentioned previously, the network biases are shown in FIG. 8(a), with the kernel weights shown in FIG. 8(b). Likewise for the second convolutional later the network biases are shown in FIG. 9(a) with the kernel weights shown in FIG. 9(b).

### REFERENCES

[1] DeNardo D G, Brennan D J, Rexhepaj E, Ruffell B, Shiao S L, Madden S F, Gallagher W M, Wadhwani N, Keil S D, Junaid S A et al: Leukocyte complexity predicts breast cancer survival and functionally regulates response to chemotherapy. Cancer Discov 2011, 1(1):54-67.

[2]. Mulrane L, Madden S F, Brennan D J, Gremel G, McGee S F, McNally S, Martin F, Crown J P, Jirstrom K, Higgins D G et al: miR-187 is an independent prognostic factor in breast cancer and confers increased invasive potential in vitro. Clin Cancer Res 2012, 18(24):6702-6713.

1. A method of determining risk of cancer recurrence in a patient, the method comprising the steps:
obtaining hyperspectral imaging training data and known recurrence outcomes for the hyperspectral imaging training data;
training one or more neural networks using the hyperspectral imaging training data and corresponding known recurrence outcomes;
obtaining hyperspectral imaging patient data from a biopsy sample obtained from the patient; and
applying the one or more neural networks to the hyperspectral imaging patient data so as to determine risk of cancer recurrence in the patient.

2. The method of claim 1, wherein the one or more neural networks are deep-learning convolutional neural networks (DL-CNN).

3. The method of claim 1, wherein the cancer is breast cancer.

4. The method of claim 3, further comprising the step of constructing tissue microarray blocks from the biopsy sample from the patients.

5. The method of any preceding claim, wherein the step of training one or more neural networks using the hyperspectral imaging training data comprises inputting the hyperspectral imaging training data as an input to the first layer of one or more of the neural networks.

6. The method of any preceding claim, wherein the hyperspectral imaging data comprise spectral data corresponding to more than 700 hyperspectral variables.

7. The method of any preceding claim, wherein the step of obtaining hyperspectral imaging patient data from a biopsy sample obtained from the patient employs an unlabeled biopsy sample.

8. The method of any preceding claim, wherein the hyperspectral imaging data is measured from formalin-fixed paraffin preserved biopsy samples.

9. The method of claim 8, wherein the formalin-fixed paraffin preserved biopsy samples are chemically dewaxed.

10. The method of any preceding claim, wherein the step of obtaining hyperspectral imaging patient data from a biopsy sample employs an infra-red imaging device.

11. The method of any preceding claim, wherein the step of obtaining hyperspectral imaging patient data from a biopsy sample employs a FTIR IR imaging device or a variant thereof.

12. The method of any of claims 1 to 9, wherein the step of obtaining hyperspectral imaging patient data from a biopsy sample employs a quantum cascade laser imaging or a variant thereof.

13. The method of any of claims 1 to 9 wherein the step of obtaining hyperspectral imaging patient data from a biopsy sample employs a Raman imaging device, stimulated Raman imaging device or coherent anti-Stokes Raman imaging device or variant thereof.

14. The method of any preceding claim, wherein the step of training the one or more neural networks comprises the steps:
using a first portion of the hyperspectral imaging training data and the corresponding known recurrence outcomes to adjust weights of the neural networks so as to produce one or more trained neural network; and
inputting a second portion of the hyperspectral imaging training data to the one or more trained neural network to validate the performance of the trained neural network in reproducing the corresponding known recurrence outcomes so as to identify a validated neural network,
and wherein the step of applying the one or more neural networks to the hyperspectral imaging patient data comprises applying the validated neural network to the hyperspectral imaging patient data so as to determine risk of cancer recurrence in the patient.

15. The method as claimed in any preceding claim wherein, the step of applying the one or more neural networks to the hyperspectral imaging patient data comprises:
inputting to a first convolution layer, images which have a predetermined dimension;
filtering the image using a plurality of first convolution layer kernels to extract features from the input image;
Sub-sampling the image to create a plurality of first feature maps;
inputting the plurality of first feature maps into a second convolution layer and filtering the feature maps using a plurality of second convolution layer kernels;
sub-sampling the output of the second convolution layer to create a plurality of second feature maps; wherein
the second feature maps is input to at least one first fully connected layer which produces a binary classification on the basis of the outputs of all of the preceding layers and which predicts recurrence or non-recurrence.

16. The method as claimed in claim 15 wherein, the image is a chemical image.

17. The method as claimed in claim 15 or claim 16 wherein, the image has a dimension of 256×256 pixels in the x-y direction and 106 wavenumbers in the z-direction.

18. The method as claimed in any of claims 15 to 17 wherein, the step of filtering the image using a plurality of first convolution layer kernels uses a layer stride is 1×1 pixel along the x-y dimension

19. The method as claimed in any of claims 15 to 18 wherein, the step of sub-sampling the image to create a plurality of first feature maps uses a max pooling layer of 2×2 pixels.

20. The method as claimed in any of claims 15 to 19 wherein, the second feature maps are processed by a first and second fully connected layer.

21. The method as claimed in any of claims 15 to 20 wherein, the first and second fully connected layers have 180 and 100 neurons, respectively.

22. The method as claimed in any of claims 15 to 20 wherein, each fully connected layer is followed by a dropout layer.

23. The method as claimed in claim 22 wherein, the dropout layer has a frequency of rate 0.5 for preventing overfitting during training.

24. The method as claimed in any preceding claim wherein the step of training and validating one or more neural network comprises:
Receiving imaging data in which recurrence of a condition was seen;
splitting the data randomly by patient into training, validation, and test data sets training the network through a predetermined number of epochs to create a plurality of models and identifying an optimal model from the models as being with model with the highest value of area under a receiver operating characteristic curve of validation.

25. A risk determination apparatus for determining risk of cancer recurrence in a patient, the risk determination apparatus comprising:
a data measurement system configured to measure hyperspectral imaging patient data;
one or more neural networks trained using hyperspectral imaging training data and corresponding known recurrence outcomes; and
a processor configured to:
receive the hyperspectral imaging patient data;
apply the one or more neural networks to the hyperspectral imaging patient data so as to determine risk of cancer recurrence in the patient.

26. A computer program product comprising a computer usable medium, where the computer usable medium comprises a computer program code that, when executed by a computer apparatus, determines risk of cancer recurrence in a patient according to the method of any of claims 1 to 24.
